(19) United States
(12) Patent Application Publication
(10) Pub. No.: US 2014/0024904 A1
(43) Pub. Date: Jan. 23, 2014

(54) BLOOD COMPONENT MEASURING DEVICE

(71) Applicant: TERUMO KABUSHIKI KAISHA, Tokyo (JP)

(72) Inventor: Masao TAKINAMI, Ashigarakami-gun (JP)

(73) Assignee: TERUMO KABUSHIKI KAISHA, Tokyo (JP)

(21) Appl. No.: 14/035,072

(22) Filed: Sep. 24, 2013

Related U.S. Application Data
(63) Continuation of application No. PCT/JP2012/055520, filed on Mar. 5, 2012.

(30) Foreign Application Priority Data
Mar. 31, 2011 (JP) 2011-078079

(51) Int. Cl. A61B 5/1455; A61B 5/1491; A61B 5/00; A61B 5/145

(52) U.S. Cl. CPC A61B 5/1455; A61B 5/14532; A61B 5/1491; A61B 5/72
USPC 600/316

(57) ABSTRACT

A blood component measuring device includes an irradiation light source configured to emit light at least in a near-infrared region, a light receiver having such sensitivity as to receive light emitted by the irradiation light source, a holding mechanism that holds and fixes a living body part, and an arithmetic device that calculates the concentration of a blood component in the living body part. The calculating means calculates the concentration of the blood component about, of the living body part, a place where the ratio of transmitted light intensity at a first wavelength relatively easily absorbed by hemoglobin and transmitted light intensity at a second wavelength relatively poorly absorbed by hemoglobin is the minimum.

### CROSS REFERENCES TO RELATED APPLICATIONS

This application is a continuation of International Application No. PCT/JP2012/055520 filed on Mar. 5, 2012, and claims priority to Japanese Application No. 2011-078079 filed on Mar. 31, 2011, the entire content of both of which is incorporated herein by reference.

### TECHNICAL FIELD

The present invention generally relates to a blood component measuring device that treats the finger of a hand or the like as a measurement site and optically measures a blood component in a non-invasive manner.

### BACKGROUND DISCUSSION

Patients with diabetes are recommended to self-measure variations in the blood glucose level on a daily basis. For example, conventionally a patient pierces a finger or the like to collect blood and measures the blood glucose level by using a measuring device. However, the above-described measurement method imposes a great burden on the patient. So, in recent years, a blood component measuring device has been developed that uses a non-invasive technique by which a blood component contained in blood can be measured through irradiation of a patient with near-infrared light.

In the measurement method used in this blood component measuring device, for example, the fact that glucose contained in blood absorbs part of near-infrared light is utilized. Specifically, part of the body of a patient (e.g. finger or the like) is irradiated with near-infrared light and the near-infrared light transmitted through the body is received, and the blood glucose level (glucose concentration) is calculated by measuring the transmittance or absorbance thereof. An example is described in Published Japanese Application Publication No. 2001-513351.

In the measurement of the blood glucose level, it is difficult to determine whether the measured transmittance or absorbance corresponds to the glucose concentration in the blood or the concentration of glucose contained in body tissue. To address this, Japanese Patent No. 3903340 utilizes pulsation of a blood vessel, and the glucose concentration in blood is calculated based on the glucose content that periodically changes.

### SUMMARY

For measurement of the blood component with relatively high accuracy, it is important to select a site (measurement site) where the blood component is richer. It is thus desirable to enhance the measurement accuracy by selecting a site where the blood component is richer than ever and performing the measurement. Disclosed here is a blood component measuring device configured to enhance the measurement accuracy by selecting a site where a blood component is richer.

According to one aspect, a blood component measuring device that irradiates a living body part with light and measures a blood component of the living body part includes an irradiation light source which emits light at least in a near-infrared region, a light receiver having sensitivity to receive light emitted by the irradiation light source, a holding mechanism that holds and fixes a position of the living body part, and calculating means for calculating a concentration of the blood component in the living body part. The calculating means calculates the concentration of the blood component at a location of the living body part in which a ratio S1/S2 of transmitted light intensity S1 at a first wavelength which is relatively easily absorbed by hemoglobin and transmitted light intensity S2 at a second wavelength which is different from the first wavelength and which is relatively poorly absorbed by hemoglobin is a minimum.

The place at which the ratio (S1/S2) of the transmitted light intensity S1 at the first wavelength which is relatively easily absorbed by hemoglobin and the transmitted light intensity S2 at the second wavelength which is relatively poorly absorbed by hemoglobin is the minimum can be considered as a place where the blood component is rich, i.e. a site where a blood vessel is present. Therefore, the concentration of the blood component is calculated at a place where the blood component is rich. Thus, the measurement accuracy of the blood component can be enhanced.

The light receiver may be a light receiving element array in which light receiving elements are arranged in a matrix, and the calculating means may calculate the concentration of the blood component about the light receiving element with which the ratio (S1/S2) of the transmitted light intensity S1 at the first wavelength and the transmitted light intensity S2 at the second wavelength is the minimum, among the light receiving elements configuring the light receiving element array. Transmitted light can thus be received by the light receiving element array at plural places of the living body part simultaneously. Therefore, the place where the ratio (S1/S2) is the minimum can be rather surely extracted with a simple device configuration.

The blood component measuring device may include a scanner mechanism that reflects light from the irradiation light source and scans the living body part with the light on an optical path between the irradiation light source and the holding mechanism. The light receiver may receive each of transmitted light with the first wavelength and transmitted light with the second wavelength about a plurality of places of the living body part by the scan with the light by the scanner mechanism. The calculating means may calculate the concentration of the blood component about the place where the ratio (S1/S2) of the transmitted light intensity S1 at the first wavelength and the transmitted light intensity S2 at the second wavelength is the minimum, of the living body part irradiated with light with the first wavelength and light with the second wavelength. According to such a configuration, the light from the irradiation light source is scanned toward the living body part by the scanner mechanism. Thus, the place where the ratio (S1/S2) is the minimum can be easily extracted although the light receiver is formed by a single light receiving element.

It is preferable for the calculating means to have a first extractor that extracts the place where the ratio (S1/S2) is the minimum in the living body part as a first measurement site, a second extractor that extracts, as a second measurement site, a place where the ratio (S1/S2) is a maximum among sites where the transmitted light intensity S2 at the second wavelength is substantially equal to the transmitted light intensity S2 of the first measurement site in the living body part, a first transmission spectrum generator that generates a transmission spectrum of the first measurement site, a second transmission spectrum generator that generates a transmission spectrum of the second measurement site, a differential transmission spectrum calculator that calculates a differential transmission spectrum between the first measurement site and the second measurement site from the transmission spectrum of the first measurement site and the transmission spectrum of the second measurement site, and a concentration calculator that calculates the concentration of the blood component based on the differential transmission spectrum.

By measuring and analyzing the differential transmission spectrum between the first measurement site and the second measurement site in this manner, the influence of the body tissue component other than blood can be corrected and the S/N ratio can be enhanced. That is, more information on blood can be acquired by eliminating the influence of the body tissue component other than blood by the correction. This can further enhance the measurement accuracy of the blood component.

Furthermore, it is preferable for the blood component measuring device to include a warming mechanism that warms the living body part. The blood flow of the living body part can be increased by warming the living body part by the warming mechanism. Therefore, it becomes easy to extract a place where the blood component is rich, which can further enhance the measurement accuracy.

According to the blood component measuring device in accordance with the present invention, the measurement accuracy can be enhanced by selecting a site where the blood component is richer and performing measurement.

According to another aspect, a blood component measuring device that irradiates a living body part with light and measures a blood component of the living body part includes: a holding mechanism that holds and fixes a position of the living body part; at least one irradiation light source which emits at different times: i) light at a first wavelength which is directed toward the living body part when the living body part is held in the holding mechanism; ii) light at a second wavelength which is directed toward the living body part when the living body part is held in the holding mechanism; and iii) light in a near-infrared region which is directed toward the living body part when the living body part is held in the holding mechanism, with the light at the first wavelength being relatively easily absorbed by hemoglobin while the light at the second wavelength is relatively poorly absorbed by hemoglobin; a light receiver which receives the light emitted by the irradiation light source; an intensity calculator calculating a transmitted light intensity S1 at the first wavelength based on output from the light receiver and calculating a transmitted light intensity S2 at the second wavelength based on output from the light receiver; a measurement site extractor which identifies as a measurement site a location on the living body part at which a ratio S1/S2 is a minimum; and calculating means for calculating a concentration of the blood component at the measurement site of the living body part.

In accordance with another aspect of the disclosure here, a method of measuring a blood component of a living body part comprises: emitting, at a plurality of locations on the living body, light possessing a first wavelength which is relatively easily absorbed by hemoglobin; emitting, at the plurality of locations on the living body, light possessing a second wavelength which is relatively poorly absorbed by hemoglobin; calculating a light intensity S1 of the light possessing the first wavelength which has passed through the living body at the plurality of locations on the living body; calculating a light intensity S2 of the light possessing the second wavelength which has passed through the living body at the plurality of locations on the living body; determining as a measurement site the location on the living body part at which a ratio S1/S2 is a minimum; emitting light in a near-infrared region at the measurement site and receiving the light in the near-infrared region that has passed through the calculating means for calculating a concentration of the blood component at the measurement site of the living body part; and measuring the blood component of the living body part based on the light in the near-infrared region that has passed through the living body part at the measurement site.

### BRIEF DESCRIPTION OF DRAWINGS

FIG. 1 is a diagram showing the schematic configuration of a blood component measuring device according to a first embodiment disclosed here.

FIG. 2 is a block diagram showing the configuration of calculating means in the blood component measuring device shown in FIG. 1.

FIG. 3 is a diagram showing the relationship between the palm temperature and the blood flow.

FIG. 4 is a flowchart showing the operation of the blood component measuring device shown in FIG. 1.

FIG. 5 is a diagram conceptually showing the position of a blood vessel in a finger.

FIG. 6 is a diagram showing the schematic configuration of a blood component measuring device according to a second embodiment disclosed here.

FIG. 7 is a diagram showing the schematic configuration of a blood component measuring device according to a third embodiment disclosed here.

FIG. 8 is a block diagram showing the configuration of calculating means in the blood component measuring device shown in FIG. 7.

FIG. 9 is a flowchart showing the operation of the blood component measuring device shown in FIG. 7.

FIG. 10 is a diagram showing the schematic configuration of a blood component measuring device according to a fourth embodiment of the present invention.

### DETALED DESCRIPTION

Set forth below, with reference to the accompanying drawing figures, is a description of embodiments of a blood component measuring device representing examples of the blood component measuring device disclosed here.

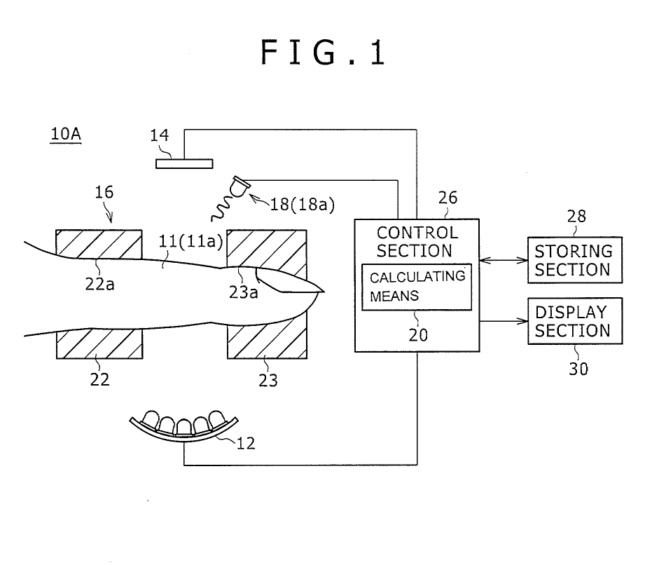

FIG. 1 schematically illustrates a blood component measuring device 10A according to a first embodiment. This blood component measuring device 10A is a medical device or medical equipment that includes an irradiation light source 12, a light receiver 14, a holding mechanism 16, a warming mechanism 18, and calculating means 20, and is configured to measure a blood component in a living body part 11 by emitting light from the irradiation light source 12 where it is transmitted through the living body part 11 so that the transmitted light is received by the light receiver 14 and then performing calculation and analysis by the calculating means 20 (calculator or calculator device) based on a signal obtained by the light receiver 14.

The living body part 11 is part of a human body and e.g. human finger 11a, palm, earlobe, etc. can be cited as examples. The blood component measuring device 10A shown in FIG. 1 is so configured as to treat the human finger 11 a as the living body part 11 and irradiate part of this finger 11a with light to measure the glucose concentration in blood at the irradiated site.

As the irradiation light source 12, a multi-wavelength LED array that can emit light in the range from visible to near-infrared and in which plural LEDs that emit light with wavelengths different from each other are arranged in a matrix for example can be employed. Furthermore, as another configuration of the irradiation light source 12, a configuration obtained by combining a light source that emits continuous light (e.g. halogen lamp) and a spectrometer (monochrometer) capable of extracting an arbitrary wavelength component may be employed.

The irradiation light source 12 can emit light with a wavelength easily absorbed by hemoglobin (first wavelength), light with a wavelength poorly absorbed by hemoglobin (second wavelength), and light in a wide wavelength region (e.g. range of about 700 nm to about 2200 nm) for acquiring the transmission spectrum of light transmitted through the living body part 11.

In skin transmission, the wavelengths easily absorbed by hemoglobin are near 760 nm and 940 nm. The wavelengths poorly absorbed by hemoglobin are wavelengths poorly absorbed also by living body tissue other than blood, among the wavelengths other than the wavelengths easily absorbed by hemoglobin, and are e.g. 1000 nm to 1300 nm. The range of 1000 nm to 1300 nm exhibits comparatively less absorption by a living body component and is referred to as the “biological window.” Glucose allows strong observation near 1600 nm although the absorption peak is not clear.

The light receiver 14 can detect light in the range from visible to near-infrared region. In the present embodiment, it is formed by a light receiving element array in which plural light receiving elements are arranged in a matrix. As such a light receiving element array, e.g. an InGaAs photodiode array is cited.

The holding mechanism 16 is configured to hold and fix the living body part 11. The holding mechanism 16 is configured to hold and fix the position of the living body part relative to the irradiation light source 12 and the irradiation light source 12. In the configuration shown in the diagram, the holding mechanism 16 is composed of two holding members 22 and 23 having holding holes 22a and 23a in which the human finger 11a is inserted. It is preferable for the holding members 22 and 23 to be formed by such an elastic member as to be elastically deformed to fit to the shape of the human finger 11 a when the finger 11a is inserted for example. As such an elastic member, e.g. an elastomer sponge or the like is cited. By forming the holding members 22 and 23 in this manner, the finger 11a can be stably held and fixed.

The warming mechanism 18 operates or functions to warm (heat) the living body part 11 in order to increase the blood flow of the living body part 11. In the configuration shown by way of example in the diagram, the warming mechanism 18 is an infrared light source 18a (e.g. infrared LED) and irradiates the part exposed between the two holding members 22 and 23, of the finger 11 a as the living body part 11, to heat this irradiated part. Other examples of the warming mechanism 18 include, for example, a configuration to warm the living body part 11 by bringing a heat source into direct contact with the living body part, a configuration to warm the living body part 11 by reducing the pressure of the living body part, a configuration to warm the living body part 11 by rubbing (massaging) the living body part, etc.

The calculating means 20 is provided as a function or a part of a control section 26 which can be in the form of a microprocessor. The control section 26, with a storing section 28, can be configured as a computer that is configured to calculate the concentration of a blood component (glucose) in the living body part 11. Thus, a microprocessor, forming part of a computer for example, is an example of structure constituting the calculating means 20 and that calculates the concentration of a blood component (glucose) in the living body part 11. A signal corresponding to the emission state of the irradiation light source 12 and a light reception signal corresponding to the intensity of transmitted light received by the light receiver 14 are input to the microprocessor. A display section 30 is provided in the blood component measuring device 10A and displays information showing the measurement result (blood glucose level) and so forth under control operation of the control section 26.

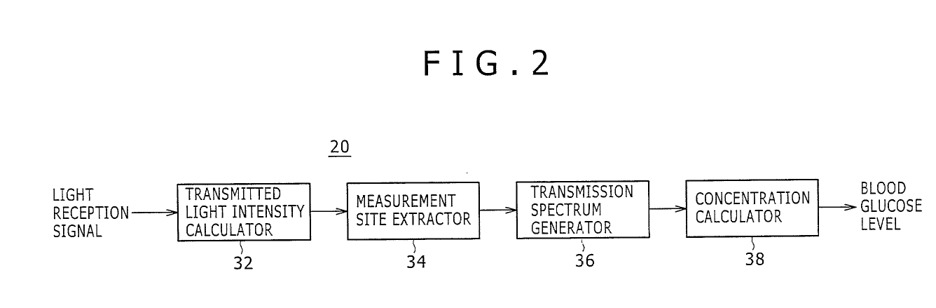

The calculating means 20 is configured to calculate the concentration of the blood component at a location or part of the living body part 11 at which the ratio (S1/S2) of transmitted light intensity S1 at the above-described first wavelength and transmitted light intensity S2 at the above-described second wavelength is the minimum. As shown in FIG. 2, the calculating means 20 has a transmitted light intensity calculator 32, a measurement site extractor 34, a transmission spectrum generator 36, and a concentration calculator 38.

The transmitted light intensity calculator 32 calculates the transmitted light intensity S1 at the first wavelength and the transmitted light intensity S2 at the second wavelength based on the light reception signals from the light receiver 14. The measurement site extractor 34 extracts the place (location or position along the finger, for example a location at which a lot of blood vessels are present) where the above-described ratio (S1/S2) is the minimum as a measurement site. The transmission spectrum generator 36 generates a transmission spectrum SP1 of the extracted measurement site. The concentration calculator 38 calculates the concentration of the blood component based on the generated transmission spectrum SP1.

In the storing section 28, a program for executing the respective kinds of processing by the transmitted light intensity calculator 32, the measurement site extractor 34, the transmission spectrum generator 36, and the concentration calculator 38 is stored. In accordance with this program, a CPU in the control section 26 executes predetermined arithmetic processing to calculate the glucose concentration by multivariate analysis or the like based on the transmitted light intensity corresponding to the light reception signal acquired by the light receiver 14, and so forth.

The blood component measuring device 10A according to the present embodiment is generally configured in the above-described manner, and the operation and effect of the blood component measuring device will be described below.

To measure the glucose concentration (blood glucose level) using the above-described blood component measuring device 10A, as shown in FIG. 1, first the human finger 11a for which the glucose concentration is to be measured is inserted in the holding mechanism 16 of the blood component measuring device 10A so that the finger is held (is held at a predetermined position). Upon the holding of the finger 11a by the holding mechanism 16, a start switch provided in the main body part of the blood component measuring device 10A is pressed to start the measurement processing.

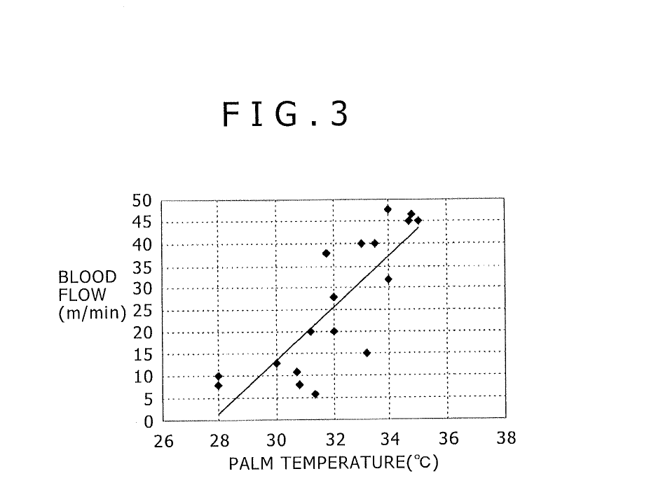

Upon the start of the measurement processing in the blood component measuring device 10A, an infrared ray is irradiated to or at the finger 11a from the infrared light source as the warming mechanism 18. Here, FIG. 3 is a diagram showing the relationship between the palm temperature and the blood flow. As is understood from FIG. 3, the blood flow increases as the palm temperature becomes higher. Therefore, the blood flow in the finger 11a can be increased by warming the finger 11a by the warming mechanism 18.

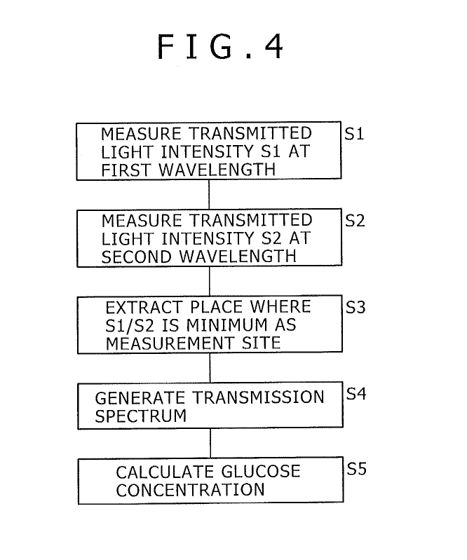

The operation of the blood component measuring device 10A will be described below with reference to the flowchart of FIG. 4.

In parallel with the warming of the finger 11a by the warming mechanism 18 or after the warming, the blood component measuring device 10A emits light at the first wavelength which is relatively easily absorbed by hemoglobin from the irradiation light source 12 and receives light transmitted through the finger 11a by the light receiver 14. The light receiver 14 outputs the light reception signal indicating the light received at the light receiver 14. The transmitted light intensity calculator 32 calculates (measures) the transmitted light intensity S1 at the first wavelength based on the light reception signal from the light receiver 14 (step S1). Furthermore, after irradiating the light at the first wavelength from the irradiation light source 12 and receiving the light reception signal from the light receiver 14 by the calculating means 20 or before irradiating the light at the first wavelength from the irradiation light source 12, the blood component measuring device 10A emits light at the second wavelength which is relatively poorly absorbed by hemoglobin from the irradiation light source 12 and receives light transmitted through the finger 11a by the light receiver 14. The light receiver 14 outputs the light reception signal indicating the light received at the light receiver 14. The transmitted light intensity calculator 32 calculates (measures) the transmitted light intensity S2 based on the light reception signal from the light receiver 14 (step S2).

Next, the measurement site extractor 34 extracts the place where the ratio (S1/S2) of the transmitted light intensity S1 at the first wavelength and the transmitted light intensity S2 at the second wavelength is the minimum as a measurement site (step S3). In the case of the present embodiment, because the light receiver 14 is formed by a light receiving element array, the light receiver 14 receives light transmitted through a certain range of the living body part 11. Therefore, specifically, the measurement site extractor 34 extracts (specifies) the light receiving element corresponding to the place where the ratio (S1/S2) is the minimum among the light receiving elements configuring the light receiving element array of the light receiver 14. That is, the light receiver 14 includes an array of light receiving elements which each receive light which has passed through a different region of the living body, and the measurement site extractor 34 extracts, specifies or identifies the light receiving element in the array at which the ratio (S1/S2) is the minimum amongst all of the light receiving elements, thus providing an indication of the region of the living body at which the ratio S1/S2 is a minimum.

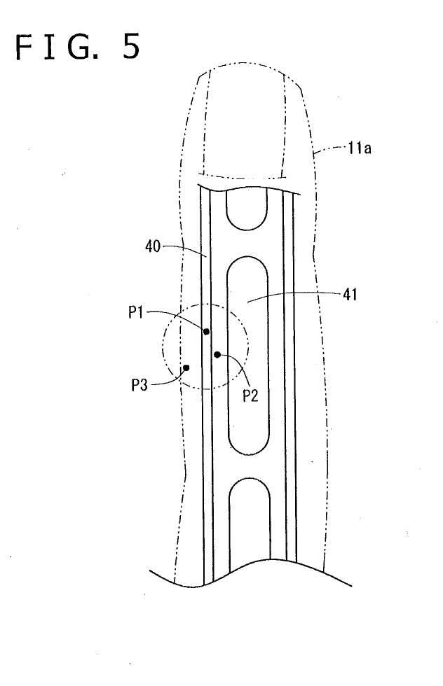

As shown in FIG. 5, the living body includes a part where the blood component richly exists (e.g., a part at which a lot of blood vessels 40 exist) and tissue components other than the blood component. The light at the first wavelength is easily absorbed by hemoglobin whereas the second wavelength is poorly absorbed by hemoglobin. Therefore, the place where the ratio (S1/S2) of the transmitted light intensity S1 at the first wavelength and the transmitted light intensity S2 at the second wavelength is a minimum can be considered as a site where the blood component is rich, i.e. a site where the blood vessel 40 is present. So, in the present invention, the place where the ratio (S1/S2) is the minimum is extracted as or determined to be a measurement site in order to enhance the measurement accuracy of the blood component. The measurement site extracted or determined in this manner is a place where the blood vessel 40 exists and is e.g. a position P1 shown in FIG. 5.

Next, the blood component measuring device 10A irradiates the finger 11 a with light in the near-infrared region by the irradiation light source 12 and receives transmitted light by the light receiver 14. That is, the blood component measuring device 10A operates so that the irradiation light source 12 emits light in the near-infrared range and irradiates the finger 11a with light in the near-infrared region, and the transmitted light passing through the living body (finger) is received by the light receiver 14. Thereupon, based on the light reception signal indicating the light received at the light receiver 14, the transmission spectrum generator 36 generates the transmission spectrum SP1 about the measurement site extracted by the measurement site extractor 34 (step S4). The transmission spectrum generator 36 which generates the transmission spectrum SP1 is a known device used in this area. Next, the concentration calculator 38 calculates the glucose concentration by multivariate analysis or the like based on the transmission spectrum SP1 generated in the transmission spectrum generator 36 (step S5). The calculation of the glucose concentration by multivariate analysis is known to ordinarily skilled artisans in this area and so a detailed description of the calculation is not provided. The display section 30 displays the glucose concentration calculated in this manner as a blood glucose level.

As described above, according to the blood component measuring device 10A in accordance with the present embodiment, the concentration of the blood component is calculated at the place where the ratio (S1/S2) of the transmitted light intensity S1 at the first wavelength which is relatively easily absorbed by hemoglobin and the transmitted light intensity S2 at the second wavelength which is relatively poorly absorbed by hemoglobin is the minimum, i.e. a place where the blood component is rich (position P1 in FIG. 5). Thus, the measurement accuracy of the blood component can be enhanced.

Furthermore, in the case of the present embodiment, the light receiving element array 14 receives transmitted light corresponding to or at plural places of the living body part 11 (finger 11a) simultaneously. Therefore, the place where the ratio (S1/S2) is the minimum can be rather surely extracted with a simple device configuration.

Moreover, in the case of the present embodiment, the living body part 11 is warmed by the warming mechanism 18. Therefore, the blood flow of the living body part 11 is increased and it becomes easier to extract or determine a place at which the blood component is rich. This can further enhance the measurement accuracy of the blood component.

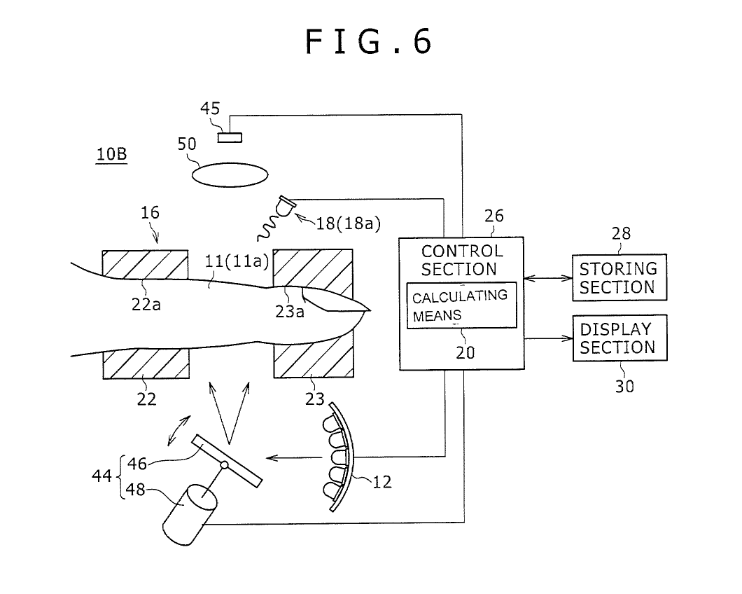

A blood component measuring device 10B according to a second embodiment will now be described with reference to FIG. 6. In the blood component measuring device 10B according to the second embodiment, features and aspects of the system that are the same as features and aspects described above are identified by common reference numerals and a detailed description of such features is not repeated.

The blood component measuring device 10B according to this second embodiment includes an irradiation light source 12, a scanner mechanism 44, a light receiver 45, a holding mechanism 16, a warming mechanism 18, and calculating means 20. The holding mechanism 16 and the calculating means 20 are formed similarly to the holding mechanism 16 and the calculating means 20 in the first embodiment.

The irradiation light source 12 has the same configuration as that of the irradiation light source 12 shown in FIG. 1. However, it is disposed at a position offset from the position opposed to a finger 11a held by the holding mechanism 16. On the emitting surface side of the irradiation light source 12, the scanner mechanism 44 that reflects light from the irradiation light source 12 and scans a living body part 11 with the light is provided. That is, the scanner mechanism 44 is disposed on or along the optical path between the irradiation light source 12 and the holding mechanism 16.

The scanner mechanism 44 includes a reflector 46 that reflects light from the irradiation light source 12 and a driver 48 that drives the reflector 46 to swing the reflector (rotate the reflector). The driver 48 and the reflector 48 are configured so that the reflector 46 rotates or swings through operation of the driver 48 to reflect light from the irradiation light source 12 and perform two-dimensional scanning with the light along the living body part 11 under operation of the control section 26.

The light receiver 45 can detect light in the range from visible to near-infrared region and is formed from a single light receiving element in the present embodiment. An example of such a light receiving element is an InGaAs photodiode. The light receiver 45 receives transmitted light at a first wavelength and transmitted light at a second wavelength at plural places of the living body part 11 in synchronization with the light scanning by the scanner mechanism 44. That is, the scanner mechanism 44 causes the transmitted light at the first wavelength to pass through different regions of the living body part 11, and the light receiver 45 receives transmitted light at the first wavelength which has passed through the living body 11. Similarly, the scanner mechanism 44 causes the transmitted light at the second wavelength to pass through different regions of the living body part 11, and the light receiver 45 receives transmitted light at the second wavelength which has passed through the living body 11.

A condenser lens 50 is disposed between the light receiver 45 and the holding mechanism 16. This condenser lens 50 condenses light transmitted through the living body part 11 toward the light receiver 45.

The calculating means 20 is configured to calculate the concentration of the blood component at the place where the ratio (S1/S2) of transmitted light intensity S1 at the first wavelength and transmitted light intensity S2 at the second wavelength received by the light receiver 45 is the minimum, of the living body part 11 irradiated with the light at the first wavelength and the light at the second wavelength. The calculating means 20 has a transmitted light intensity calculator 32, a measurement site extractor 34, a transmission spectrum generator 36, and a concentration calculator 38 similar to the calculating means 20 shown in FIG. 2.

To measure the glucose concentration (blood glucose level) by the above-described blood component measuring device 10B, first the human finger 11a about which the glucose concentration is to be measured is inserted in the holding mechanism 16 of the blood component measuring device 10B to be held at a predetermined position. Upon the holding of the finger 11a by the holding mechanism 16, a start switch provided in the main body part, of the blood component measuring device 10B is pressed or operated to start the measurement processing. Thereupon, an infrared ray is irradiated toward the finger 11a from an infrared light source as the warming mechanism 18, so that the living body part 11 is warmed.

In parallel with (i.e., during) the warming of the finger 11a by the warming mechanism 18 or after the warming, the blood component measuring device 10B operates so that the irradiation light source 12 emits light at the first wavelength which is relatively easily absorbed by hemoglobin, and the light receiver 45 receives light at the first wavelength transmitted through the finger 11a from the irradiation light source 12. The transmitted light intensity calculator 32 calculates (measures) the transmitted light intensity S1 at the first wavelength based on a light reception signal from the light receiver 45. Furthermore, after irradiating the light with the first wavelength from the irradiation light source 12 and receiving the light reception signal from the light receiver 45 by the calculating means 20 or before irradiating the light with the first wavelength from the irradiation light source 12, the blood component measuring device 10B operates so that the irradiation light source 12 emits light at the second wavelength which is relatively poorly absorbed by hemoglobin and the light receiver 45 receives light at the second wavelength transmitted through the finger 11a from the irradiation light source 12. This light reception signal is sent to the calculating means 20. Thereupon, the transmitted light intensity calculator 32 calculates (measures) the transmitted light intensity S2 based on the light reception signal from the light receiver 45.

In this embodiment, the system is configured so that the light from the irradiation light source 12 is reflected by the scanner mechanism 44 and scanning is performed. In addition, the light receiver 45 is formed by a single light receiving element. Therefore, by associating the scanning position of the scanner mechanism 44 with the light reception signal from the light receiver 45, the transmitted light intensity at each of plural places along the living body part 11 can be calculated.

Next, the measurement site extractor 34 extracts, as a measurement site, the place where the ratio (S1/S2) of the transmitted light intensity S1 at the first wavelength and the transmitted light intensity S2 at the second wavelength is the minimum (place indicated by P1 in FIG. 5), of the living body part 11 irradiated with the light with the first wavelength and the light with the second wavelength.

Next, the blood component measuring device 10B irradiates the finger 11a with light in the near-infrared region by the irradiation light source 12 and receives transmitted light thereof by the light receiver 45. At this time, the operating position of the scanner mechanism 44 is so controlled that the extracted measurement site (e.g., P1) is irradiated with the light from the irradiation light source 12, and light transmitted through the measurement site is condensed by the condenser lens 50 to be received by the light receiver 45. Thereupon, based on the light reception signal indicating the light received at the light receiver 45, the transmission spectrum generator 36 generates a transmission spectrum SP1 about the measurement site extracted by the measurement site extractor 34.

Next, the concentration calculator 38 calculates the glucose concentration by multivariate analysis or the like based on the transmission spectrum SP1 generated in the transmission spectrum generator 36. The display section 30 displays the glucose concentration calculated in this manner as a blood glucose level.

As described above, according to the blood component measuring device 10B in accordance with the present embodiment, the concentration of the blood component is calculated at the place where the ratio (S1/S2) of the transmitted light intensity S1 at the first wavelength relatively easily absorbed by hemoglobin and the transmitted light intensity S2 at the second wavelength relatively poorly absorbed by hemoglobin is the minimum, i.e. a place where the blood component is rich. Thus, the measurement accuracy of the blood component can be enhanced.

Furthermore, in the case of the present embodiment, the light from the irradiation light source 12 is scanned toward the living body part 11 by the scanner mechanism 44. Thus, the place where the ratio (S1/S2) is the minimum can be relatively easily extracted although the light receiver 45 is formed by a single element.

In the second embodiment, the respective constituent parts in common with those in the first embodiment provide the same or similar operation and effect as those provided by these common respective constituent parts in the first embodiment.

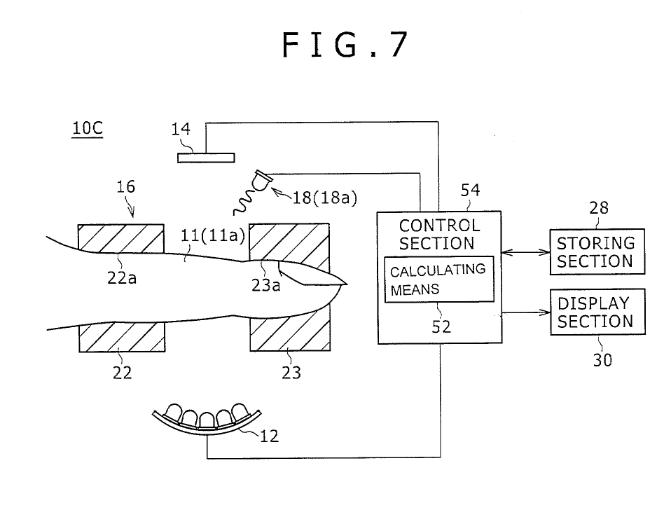

FIG. 7 is a diagram showing the schematic configuration of a blood component measuring device 10C according to a third embodiment disclosed here. This blood component measuring device 10C is medical equipment or a medical device that includes an irradiation light source 12, a light receiver 14, a holding mechanism 16, a warming mechanism 18, and calculating means 52, and is configured to measure a blood component in a living body part 11 by transmitting light emitted from the irradiation light source 12 through the living body part 11 to receive the transmitted light by the light receiver 14 and performing calculation and analysis by the calculating means 52 of a signal obtained by the light receiver 14.

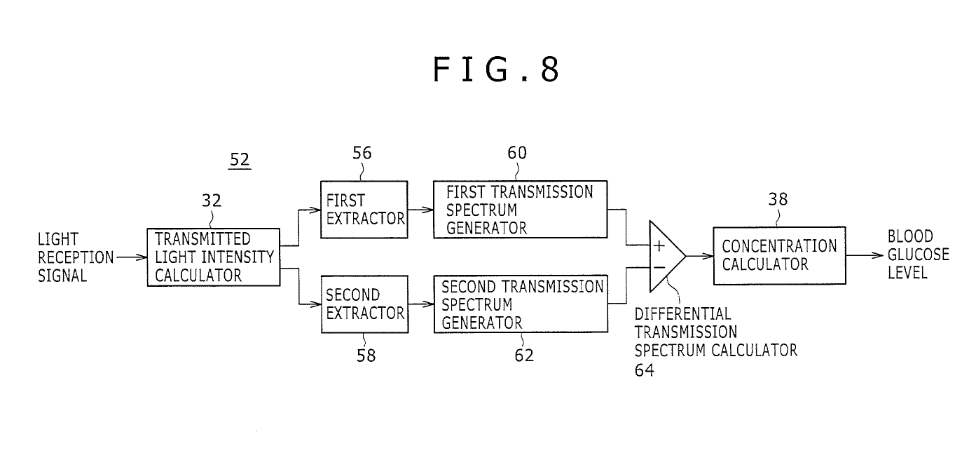

The blood component measuring device 10C according to the present embodiment differs from the blood component measuring device 10A according to the first embodiment in the configuration of the calculating means 52. Specifically, as shown in FIG. 8, the calculating means 52 has a transmitted light intensity calculator 32, a first extractor 56, a second extractor 58, a first transmission spectrum generator 60, a second transmission spectrum generator 62, a differential transmission spectrum calculator 64, and a concentration calculator 38.

The transmitted light intensity calculator 32 calculates transmitted light intensity S1 at a first wavelength and transmitted light intensity S2 at a second wavelength. The first extractor 56 extracts the place of the living body part 11 at which the above-described ratio (S1/S2) is the minimum, and this place is identified as a measurement site (hereinafter, referred to as first measurement site). The second extractor 58 extracts, as a second measurement site, the place that is a site where the transmitted light intensity S2 at the second wavelength is substantially equal to the transmitted light intensity S2 at the second wavelength of the first measurement site and where the ratio (S1/S2) is the maximum. The first transmission spectrum generator 60 generates a transmission spectrum SP1 of the first measurement site. The second transmission spectrum generator 62 generates a transmission spectrum SP2 of the second measurement site. The differential transmission spectrum calculator 64 calculates a differential transmission spectrum dSP (=SP1−SP2) between the first measurement site and the second measurement site. The concentration calculator 38 calculates the concentration of the blood component based on the differential transmission spectrum dSP.

In the storing section 28 connected to the calculating means 52, a program for executing the respective kinds of processing by the transmitted light intensity calculator 32, the first extractor 56, the second extractor 58, the first transmission spectrum generator 60, the second transmission spectrum generator 62, and the differential transmission spectrum calculator 64 is stored. In accordance with this program, a CPU in a control section 54 executes predetermined arithmetic processing to calculate the glucose concentration by multivariate analysis or the like based on the transmitted light intensity acquired by the light receiver 14, and so forth.

The irradiation light source 12 and the warming mechanism 18 are controlled by the control section 54 which can be in the form of a microprocessor. The control section 54, with a storing section 28, can be configured as a computer that is configured to calculate the concentration of a blood component (glucose) in the living body part 11. Thus, a microprocessor, forming part of a computer for example, is an example of structure constituting the calculating means 52 and that calculates the concentration of a blood component (glucose) in the living body part 11.

To measure the glucose concentration (blood glucose level) by the above-described blood component measuring device 100, first a human finger 11 a at which the glucose concentration is to be measured is inserted in the holding mechanism 16 of the blood component measuring device 10C to be held at a predetermined position. When the finger 11a is held by the holding mechanism 16, a start switch provided in the main body part of the blood component measuring device 10C is pressed or operated to start the measurement processing. Thereupon, an infrared ray is irradiated toward the finger 11a from an infrared light source as the warming mechanism 18, so that the living body part 11 is warmed.

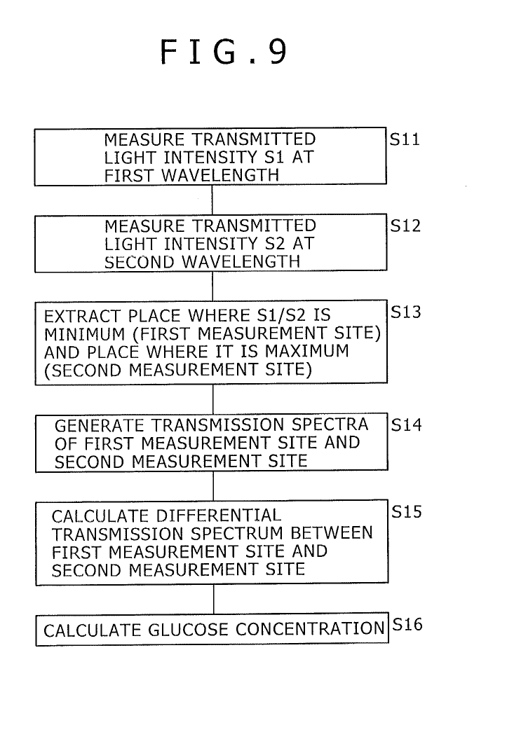

The operation of the blood component measuring device 10C will now be described below with reference to a flowchart of FIG. 9.

In parallel with the warming of the finger 11a by the warming mechanism 18 or after the warming, light at the first wavelength which is relatively easily absorbed by hemoglobin is emitted by the irradiation light source 12 of the blood component measuring device 10C, and light transmitted through the finger 11a is received by the light receiver 14 of the blood component measuring device 100. The light receiver 14 outputs a light reception signal indicating the light received at the light receiver 14. The transmitted light intensity calculator 32 calculates (measures) the transmitted light intensity S1 based on the light reception signal from the light receiver 14 (step S11). Furthermore, after irradiating or emitting the light at the first wavelength from the irradiation light source 12 and after the calculating means 52 receives the light reception signal from the light receiver 14 or before irradiating or emitting the light at the first wavelength from the irradiation light source 12, the irradiation light source 12 of the blood component measuring device 10C emits light at the second wavelength which is relatively poorly absorbed by hemoglobin, and the light receiver 14 receives light transmitted through the finger 11a. The light receiver 14 outputs the light reception signal indicating the light received at the light receiver 14. Thereupon, the transmitted light intensity calculator 32 calculates (measures) the transmitted light intensity S2 based on the light reception signal from the light receiver 14 (step S12).

Next, by the first extractor 56, the place where the ratio (S1/S2) of the transmitted light intensity S1 at the first wavelength and the transmitted light intensity S2 at the second wavelength is the minimum, of the living body part 11, is extracted as a first measurement site (step S13). The extracted first measurement site is a place where the blood vessel 40 exists and is e.g. the position P1 shown in FIG. 5. In the case of the present embodiment, because the light receiver 14 is formed by a light receiving element array, the light receiver 14 receives light transmitted through a certain range of the living body part 11. Therefore, specifically, the first extractor 56 extracts (specifies) the light receiving element corresponding to the place where the ratio (S1/S2) is the minimum among the light receiving elements configuring or forming the light receiving element array of the light receiver 14.

Furthermore, the second extractor 58 extracts the place where the ratio (S1/S2) is the maximum among sites where the transmitted light intensity S2 at the second wavelength is substantially equal to that of the first measurement site in the living body part 11 (step S13). In the case of the present embodiment, specifically, the second extractor 58 extracts (specifies) the light receiving element corresponding to the place where the ratio (S1/S2) is the maximum among places where the transmitted light intensity S2 at the second wavelength is substantially equal to that of the first measurement site in the light receiving elements configuring the light receiving element array of the light receiver 14.

Even light transmitted through the blood vessel 40 is inevitably transmitted through a body tissue component other than blood. This causes a measurement error due to the influence of the body tissue component other than blood. Therefore, it is preferable to remove the influence of the body tissue component other than blood to reduce the measurement error. However, the measurement error is not necessarily reduced by simply eliminating the influence of the body tissue component at the part spaced from the blood vessel 40.

For example, in FIG. 5, amongst places where the blood vessel 40 does not exist on the transmission path of light, at a side end part P3 of the finger 11a, the body transmission distance of transmitted light is considerably shorter compared with that at a place where the blood vessel 40 exists. Therefore, the measurement error cannot be effectively reduced even when the influence of the body tissue component at the side end part P3 is eliminated. Furthermore, even when the influence of a place where a bone 41 exists is eliminated, the measurement error cannot be effectively reduced.

So, in the present embodiment, the place where the above-described ratio (S1/S2) is the maximum among places where the transmitted light intensity S2 at the second wavelength is substantially equal to that of the first measurement site is extracted as the second measurement site to eliminate the influence of the body tissue component at the second measurement site. The second measurement site extracted in this manner is e.g. a position P2 near the blood vessel 40 shown in FIG. 5. This second measurement site is substantially equal to the first measurement site in the body passage distance of transmitted light. Therefore, the measurement error can be effectively reduced by eliminating the influence of the body tissue component at this part.

Next, the irradiation light source 12 of the blood component measuring device 10C emits light in the near-infrared range or irradiates the finger 11a with light in the near-infrared region, and the light receiver 14 receives transmitted light. Thereupon, based on the light reception signal thereof, the first transmission spectrum generator 60 generates the transmission spectrum SP1 of light transmitted through the first measurement site and the second transmission spectrum generator 62 generates the transmission spectrum SP2 of light transmitted through the second measurement site (step S14).

Next, the differential transmission spectrum calculator 64 calculates the differential transmission spectrum dSP (=SP1−SP2) between the transmission spectrum SP1 of the first measurement site and the transmission spectrum SP2 of the second measurement site (step S15). Next, the concentration calculator 38 calculates the concentration of the blood component (glucose) by multivariate analysis or the like based on the calculated differential transmission spectrum dSP (step S16). The display section 30 displays the glucose concentration calculated in this manner as a blood glucose level.

As described above, according to the blood component measuring device 10C in accordance with the present embodiment, the concentration of the blood component is calculated at the place where the ratio (S1/S2) of the transmitted light intensity S1 at the first wavelength which is relatively easily absorbed by hemoglobin and the transmitted light intensity S2 at the second wavelength which is relatively poorly absorbed by hemoglobin is the minimum, i.e. a place where the blood component is rich. Thus, the measurement accuracy of the blood component can be enhanced.

Furthermore, in the case of the present embodiment, the influence of the body tissue component other than blood can be corrected and the S/N ratio can be enhanced by measuring and analyzing the differential transmission spectrum dSP between the first measurement site and the second measurement site. That is, more information on blood can be acquired by eliminating the influence of the body tissue component other than blood by the correction. This can further enhance the measurement accuracy of the blood component.

In this third embodiment, the respective constituent parts that are common with parts in the first embodiment achieve the same or similar operation and effect as those provided by these common respective constituent parts in the first embodiment.

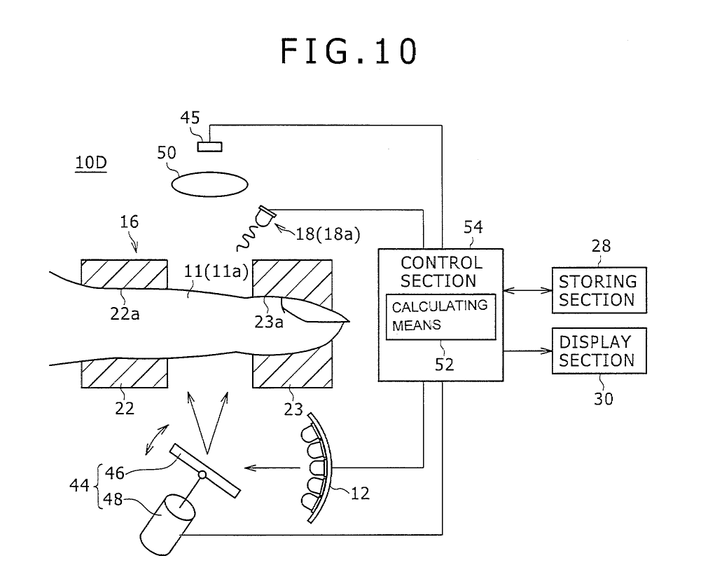

Next, a blood component measuring device 10D according to a fourth embodiment will be described with reference to FIG. 10. In the blood component measuring device 10D according to the fourth embodiment, features and aspects of the system that are the same as features and aspects of the third embodiment of the blood component measuring device 10C described above are identified by common reference numerals and a detailed description of such features is not repeated.

The blood component measuring device 10D according to the present embodiment includes an irradiation light source 12, a scanner mechanism 44, a light receiver 45, a holding mechanism 16, a warming mechanism 18, and calculating means 52. The irradiation light source 12, the holding mechanism 16, and the warming mechanism 18 are formed similar to the irradiation light source 12, the holding mechanism 16, and the warming mechanism 18 in the first embodiment. The scanner mechanism 44 is formed similar to the scanner mechanism 44 in the second embodiment. The calculating means 52 has a transmitted light intensity calculator 32, a first extractor 56, a second extractor 58, a first transmission spectrum generator 60, a second transmission spectrum generator 62, a differential transmission spectrum calculator 64, and a concentration calculator 38 similar to the calculating means 52 in the third embodiment (see FIG. 8).

To measure the glucose concentration (blood glucose level) by the above-described blood component measuring device 10D, first a human finger 11a about which the glucose concentration is to be measured is inserted in the holding mechanism 16 of the blood component measuring device 10D to be held at a predetermined position. With the finger 11a held by the holding mechanism 16, a start switch provided in the main body part of the blood component measuring device 10D is pressed or operated to start the measurement processing. Thereupon, an infrared ray is irradiated toward the finger 11a from an infrared light source as the warming mechanism 18, so that the living body part 11 is warmed.

In parallel with the warming of the finger 11a by the warming mechanism 18 or after the warming, the irradiation light source 12 of the blood component measuring device 10D emits light at a first wavelength which is relatively easily absorbed by hemoglobin, and the light receiver 45 of the blood component measuring device receives light transmitted through the finger 11a. The transmitted light intensity calculator 32 calculates (measures) transmitted light intensity S1 at the first wavelength based on a light reception signal from the light receiver 45 indicating the light received at the light receiver 45. Furthermore, after the irradiation light source 12 irradiates or emits light at the first wavelength and after the calculating means 52 receives the light reception signal from the light receiver 45 or before the irradiation light source 12 irradiates or emits the light at the first wavelength from the irradiation light source 12, the irradiation light source 12 of the blood component measuring device 10D emits light at a second wavelength which is relatively poorly absorbed by hemoglobin, and the light receiver 45 receives light transmitted through the finger 11a. The light receiver 45 outputs the light reception signal indicating the light received at the light receiver 45. The transmitted light intensity calculator 32 calculates (measures) transmitted light intensity S2 based on the light reception signal from the light receiver 45.

Next, the first extractor 56 extracts, as a first measurement site, the place of the living body part 11 irradiated with the light at the first wavelength and the light at the second wavelength at which the ratio (S1/S2) of the transmitted light intensity S1 at the first wavelength and the transmitted light intensity S2 at the second wavelength is the minimum. The second extractor 58 extracts, as a second measurement site, the place in the living body part 11 irradiated with the light at the first wavelength and the light at the second wavelength at which the ratio (S1/S2) is the maximum among sites where the transmitted light intensity S2 at the second wavelength is substantially equal to that of the first measurement site.

Next, in order to generate transmission spectra SP1 and SP2 of light transmitted through the first measurement site and the second measurement site, respectively, the irradiation light source 12 of the blood component measuring device 10D irradiates the finger 11a with light in the near-infrared region, and the light receiver 45 receives transmitted light thereof. At this time, first the operating position of the scanner mechanism 44 is so controlled that the first measurement site is irradiated with the light from the irradiation light source 12, and light transmitted through the first measurement site is received by the light receiver 45. Thereupon, based on the light reception signal thereof, the first transmission spectrum generator 60 generates the transmission spectrum SP1 at the first measurement site.

Next, the operating position of the scanner mechanism 44 is so controlled that the second measurement site is irradiated with the light from the irradiation light source 12, and light transmitted through the second measurement site is received by the light receiver 45. Thereupon, based on the light reception signal thereof, the second transmission spectrum generator 62 generates the transmission spectrum SP2 about the second measurement site.

In the acquisition of the transmission spectra SP1 and SP2, differently from the above-described operation order, the processing of irradiating the second measurement site with near-infrared light to generate the transmission spectrum SP2 of the second measurement site from transmitted light thereof may be executed before the processing of irradiating the first measurement site with near-infrared light to generate the transmission spectrum SP1 of the first measurement site from transmitted light thereof.

Next, the differential transmission spectrum calculator 64 calculates the differential transmission spectrum dSP (=SP1−SP2) between the transmission spectrum SP1 of the first measurement site and the transmission spectrum SP2 of the second measurement site. Next, the concentration calculator 38 calculates the concentration of the blood component (glucose) by multivariate analysis or the like based on the calculated differential transmission spectrum dSP. The display section 30 displays the glucose concentration calculated in this manner as a blood glucose level.

As described above, according to the blood component measuring device 10D, similar to the blood component measuring devices 10A-10C in accordance with the first to third embodiments, the concentration of the blood component is calculated at the place where the ratio (S1/S2) of the transmitted light intensity S1 at the first wavelength which relatively easily absorbed by hemoglobin and the transmitted light intensity S2 at the second wavelength which is relatively poorly absorbed by hemoglobin is the minimum, i.e. a place where the blood component is rich. Thus, the measurement accuracy of the blood component can be enhanced.

Furthermore, similar to the blood component measuring device 10B in accordance with the second embodiment, the light from the irradiation light source 12 is scanned toward the living body part 11 by the scanner mechanism 44. Thus, the place where the ratio (S1/S2) is the minimum can be easily extracted although the light receiver 45 is formed by a single element.

Moreover, like the blood component measuring device 10C in accordance with the third embodiment, the influence of the body tissue component other than blood can be corrected and the SIN ratio can be enhanced by measuring and analyzing the differential transmission spectrum dSP between the first measurement site and the second measurement site. That is, more information on blood can be acquired by eliminating the influence of the body tissue component other than blood by the correction. This can further enhance the measurement accuracy of the blood component.

The detailed description above describes features and aspects of embodiments, disclosed by way of example, of a blood component measuring device. The invention is not limited, however, to the precise embodiments and variations described and illustrated. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. A blood component measuring device that irradiates a living body part with light and measures a blood component of the living body part, the blood component measuring device comprising:
a holding mechanism that holds and fixes a position of the living body part;
at least one irradiation light source which emits at different times: i) light at a first wavelength which is directed toward the living body part when the living body part is held in the holding mechanism; ii) light at a second wavelength which is directed toward the living body part when the living body part is held in the holding mechanism; and iii) light in a near-infrared region which is directed toward the living body part when the living body part is held in the holding mechanism, the light at the first wavelength being relatively easily absorbed by hemoglobin while the light at the second wavelength is relatively poorly absorbed by hemoglobin;
a light receiver which receives the light emitted by the irradiation light source;
an intensity calculator calculating a transmitted light intensity S1 at the first wavelength based on output from the light receiver and calculating a transmitted light intensity S2 at the second wavelength based on output from the light receiver;
a measurement site extractor which identifies as a measurement site a location on the living body part at which a ratio S1/S2 is a minimum; and
calculating means for calculating a concentration of the blood component at the measurement site of the living body part.

2. The blood component measuring device according to claim 1, wherein the light receiver is a light receiving element array in which a plurality of light receiving elements are arranged in a matrix.

3. The blood component measuring device according to claim 2, wherein the calculating means calculates the concentration of the blood component at one of the light receiving elements, from amongst the plurality of light receiving elements forming the light receiving element array.

4. The blood component measuring device according to claim 1, further comprising a scanner mechanism, positioned between the at least one irradiation light source and the holding mechanism, that reflects light from the irradiation light source and scans the living body part with the light along an optical path between the irradiation light source and the holding mechanism.

5. The blood component measuring device according to claim 4, wherein the light receiver receives the transmitted light with the first wavelength and transmitted light with the second wavelength at a plurality of places of the living body part by scanning of the light by the scanner mechanism.

6. The blood component measuring device according to claim 1, wherein the calculating means includes:
a first extractor that extracts, as a first measurement site, the location on the living body at which the ratio (S1/S2) is the minimum;
a second extractor that extracts, as a second measurement site, a place on the living body at which the ratio (S1/S2) is a maximum among sites where the transmitted light intensity S2 at the second wavelength is substantially equal to the transmitted light intensity S1 of the first measurement site;
a first transmission spectrum generator that generates a transmission spectrum of the first measurement site;
a second transmission spectrum generator that generates a transmission spectrum of the second measurement site;
a differential transmission spectrum calculator that calculates a differential transmission spectrum between the first measurement site and the second measurement site from the transmission spectrum of the first measurement site and the transmission spectrum of the second measurement site; and
a concentration calculator that calculates the concentration of the blood component based on the differential transmission spectrum.

7. The blood component measuring device according to claim 1, further comprising a warming mechanism that warms the living body part.

8. A blood component measuring device that irradiates a living body part with light and measures a blood component of the living body part, the blood component measuring device comprising an irradiation light source which emits light at least in a near-infrared region;
a light receiver having sensitivity to receive light emitted by the irradiation light source;
a holding mechanism that holds and fixes a position of the living body part;
calculating means for calculating a concentration of the blood component in the living body part;
the calculating means calculating the concentration of the blood component at a location of the living body part in which a ratio S1/S2 of transmitted light intensity S1 at a first wavelength which is relatively easily absorbed by hemoglobin and transmitted light intensity S2 at a second wavelength which is different from the first wavelength and which is relatively poorly absorbed by hemoglobin is a minimum.

9. The blood component measuring device according to claim 8, wherein the light receiver is a light receiving element array in which light receiving elements are arranged in a matrix, and the calculating means calculating means calculates the concentration of the blood component at one of the light receiving elements, from amongst the plurality of light receiving elements forming the light receiving element array.

10. The blood component measuring device according to claim 8, further comprising a scanner mechanism, positioned between the at least one irradiation light source and the holding mechanism, that reflects light from the irradiation light source and scans the living body part with the light along an optical path between the irradiation light source and the holding mechanism;
wherein the light receiver receives, at a plurality of places of the living body part, both the transmitted light at the first wavelength and the transmitted light at the second wavelength through scanning of the light as a result of operation of the scanner mechanism; and
wherein the calculating means calculates the concentration of the blood component at the place of the living body part which is irradiated with light at the first wavelength and light at the second wavelength where the ratio of the transmitted light intensity S1 at the first wavelength and the transmitted light intensity S2 at the second wavelength is the minimum.

11. The blood component measuring device according to claim 8, wherein the calculating means includes:
a first extractor that extracts, as a first measurement site, the location on the living body at which the ratio (S1/S2) is the minimum;
a second extractor that extracts, as a second measurement site, a place on the living body at which the ratio (S1/S2) is a maximum among sites where the transmitted light intensity S2 at the second wavelength is substantially equal to the transmitted light intensity S1 of the first measurement site;
a first transmission spectrum generator that generates a transmission spectrum of the first measurement site;
a second transmission spectrum generator that generates a transmission spectrum of the second measurement site;
a differential transmission spectrum calculator that calculates a differential transmission spectrum between the first measurement site and the second measurement site from the transmission spectrum of the first measurement site and the transmission spectrum of the second measurement site; and
a concentration calculator that calculates the concentration of the blood component based on the differential transmission spectrum.

12. The blood component measuring device according to claim 8, further comprising a warming mechanism that warms the living body part.

13. A method of measuring a blood component of a living body part comprising:
emitting, at a plurality of locations on the living body, light possessing a first wavelength which is relatively easily absorbed by hemoglobin;
emitting, at the plurality of locations on the living body, light possessing a second wavelength which is relatively poorly absorbed by hemoglobin;
calculating a light intensity S1 of the light possessing the first wavelength which has passed through the living body at the plurality of locations on the living body;
calculating a light intensity S2 of the light possessing the second wavelength which has passed through the living body at the plurality of locations on the living body;
determining as a measurement site the location on the living body part at which a ratio S1/S2 is a minimum;
emitting light in a near-infrared region at the measurement site and receiving the light in the near-infrared region that has passed through the calculating means for calculating a concentration of the blood component at the measurement site of the living body part; and
measuring the blood component of the living body part based on the light in the near-infrared region that has passed through the living body part at the measurement site.

14. The method according to claim 13, further comprising warming the living part before emitting the light possessing the first wavelength and before emitting the light possessing the second wavelength.
